(19) United States
(12) Patent Application Publication
(10) Pub. No.: US 2024/0369476 A1
(43) Pub. Date: Nov. 7, 2024

(54) PRESENCE ENCODING TAGGANTS WITH SUPERIMPOSED ENCODING

(71) Applicant: MICROSOFT TECHNOLOGY LICENSING, LLC, Redmond, WA (US)

(72) Inventors: Yuan-Jyue CHEN, Seattle, WA (US); Sergey YEKHANIN, Redmond, WA (US); Alexander Steven CROWN, Bellevue, WA (US)

(21) Appl. No.: 18/516,485

(22) Filed: Nov. 21, 2023

Related U.S. Application Data
(60) Provisional application No. 63/463,884, filed on May 3, 2023.

(51) Int. Cl. G01N 21/31; G16C 20/20

(52) U.S. Cl. CPC G01N 21/31; G16C 20/20

(57) ABSTRACT

Molecular taggants are used to label items with binary barcodes. Molecular taggants may use presence encoding with the presence or absence of a particular molecule representing a 0 or 1. Molecular taggants may alternatively use hybridization encoding where the hybridization of a second strand to a polynucleotide represents a 0 or 1. If multiple such molecular taggants are combined, this leads to a bitwise OR operation that may make it impossible to correctly identify the original binary barcodes. To address this problem, superimposed encoding is applied to the design of the binary barcodes. The superimposed encoding identifies a set of valid binary barcodes that can be uniquely identified from a sample comprising a mixture molecular taggants. One way of doing this is to generate a Hadamard matrix and use the values of entries in the matrix as one of the binary barcodes.

### PRIORITY APPLICATION

This application claims the benefit of and priority to U.S. Provisional Application No. 63/463,884, filed May 3, 2023, the entire contents of which are incorporated herein by reference.

### BACKGROUND

Molecular taggants have great potential in supply chain tracking. The nano-scale size makes it possible to label almost any item regardless of its form factor. This makes molecular taggants useful for tracking raw materials, food, and other items. Each molecular taggant encodes some type of information associated with the item. For example, a molecular taggant could encode the name of a factory or farm that produced the item. Sometimes in supply chains, multiple molecular taggants are applied at the same item or separately labeled items are mixed together. One example is an item moving through a supply chain that is tagged with a separate molecular taggant at different stages in the supply chain. Another example is an item that is combined with other items, each individually tagged, to create a mixed item. For example, a bag of salad mix can be created by combining lettuce from multiple different sources.

Some types of molecular taggants work well if used individually but may be problematic if combined. Once combined, the molecules of distinct molecular taggants are mixed and reading mixed molecular taggants may fail to accurately recover the information encoded by the original molecular taggants. Accordingly, it is desirable to create molecular taggants that allow the information encoded by each to be recovered from a mixed sample. The following disclosure is made with respect to these and other considerations.

### SUMMARY

This disclosure provides a solution that uses superimposed encoding with molecular taggants so that the original molecular taggants can be identified even if multiple taggants are mixed together. The inability to correctly decode mixed samples arises for molecular taggants that use an encoding scheme that may result in bit errors when multiple taggants are mixed. One encoding scheme with this characteristic is presence encoding. Presence encoding uses the presence or absence of a particular molecule to represent a bit. For example, if a molecular taggant encodes a binary barcode such as 0110, each position from 1 to 4 in that binary barcode is represented by a different molecule. For positions in which there is a 0, the corresponding molecule is not included. The corresponding molecule is included for positions in which there is a 1. Thus, this molecular taggant would consist of two different molecules one to represent the 1 at the second position and one to represent the 1 at the third position. A second molecular taggant that uses the same presence encoding scheme can represent a different binary barcode such as 1001. This is encoded by a different combination of molecules. If these two molecular taggants are mixed, the corresponding four molecules present on the item would appear to encode the binary barcode 1111. This would be incorrect. The distinction between 0110 and 1001 would be lost.

The superimposed encoding creates a relationship between all valid binary barcodes—specifically that when molecular taggants encoding those binary barcodes are mixed, reading of the molecules in a sample can be mapped back to the original binary barcodes. One way of doing this is to use entries (e.g., columns or rows) of a Hadamard matrix as the binary barcodes. A Hadamard matrix is a square matrix in which each value is either 0 or 1 and whose columns and rows are mutually orthogonal. Thus, for example, a 16×16 Hadamard matrix can be used to generate 16 different binary barcodes that each include 16 bits.

The molecules used to create a molecular taggant can be synthesized in advance efficiently and economically. These molecules may be polynucleotide strands with specific sequences, fluorophores, radioactive isotopes, small organic molecules, or any type of molecule that can be uniquely identified. The number of different molecules used for a presence encoding scheme depends on the size of the binary barcode. If an eight-bit binary barcode is used, eight distinct molecules are needed to create all possible molecular taggants according to that presence encoding scheme. A given item is labeled with a particular binary barcode based on the selection of which of the eight molecules (i.e., those corresponding to positions in which the bit value is 1) are applied to the item. The combinations of which individual molecules can be combined to create a valid binary barcode are governed by the superimposed encoding.

At some point in a supply chain, a sample of one or more molecular taggant(s) is collected from an item. That sample is read using a technique appropriate for the specific type of molecules that make up the molecular taggant(s). Thus, the readout process detects individual molecules that come from the molecular taggant(s) applied to the item. There is two-sided error in the readout process. Molecules that should be present may not be detected and the readout may appear to detect molecules that are absent. The readout is an error-prone readout. This adds an additional challenge to identifying the original molecular taggants. Not only is there the challenge of separately identifying a mixture of different binary barcodes created through presence encoding, but the identification should be performed in a way that is robust enough to tolerate some degree of error.

The act of decoding an error-prone readout into one or more binary barcodes can be done by use of a lookup table. The lookup table can include entries for each of the valid binary barcodes and may also include entries for possible readouts that result from a combination of multiple ones of the binary barcodes. The lookup table is designed so that each entry is sufficiently different from all other entries to enable mapping a readout to a single entry even in the presence of errors. Thus, even if an error-prone readout does not match any entry in the lookup table exactly because of errors, the most similar entry can be identified and used to determine the barcode(s) that are represented in the sample collected from the item. Accordingly, this technique makes it possible to read and individually identify mixed molecular taggants that use presence encoding even in the presence of errors.

This Summary is provided to introduce a selection of concepts in a simplified form that are further described below in the Detailed Description. This Summary is not intended to identify key features or essential features of the claimed subject matter nor is it intended to be used to limit the scope of the claimed subject matter. The term “techniques,” for instance, may refer to system(s) and/or method(s) as permitted by the context described above and throughout the document.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. The figures are schematic representations and items shown in the figures are not necessarily to scale.

FIG. 1 illustrates a technique for tagging one or more items with molecular taggants reading mixed molecular taggants.

FIG. 2 illustrates a lookup table and a barcode correspondence table.

FIG. 3 is a flow diagram showing an illustrative method for applying molecular taggants to one or more items, reading the molecular taggants, and displaying human-readable identifiers corresponding to the taggants.

FIG. 4 is a flow diagram showing an illustrative method for designing and creating molecular taggants that can be read and decoded when mixed.

FIG. 5 is an illustrative computer architecture for implementing techniques of this disclosure.

### DETAILED DESCRIPTION

This disclosure provides a system and technique for using molecular taggants based on encoding that could lead to bit errors if multiple taggants are mixed. The techniques of this disclosure provide a way that multiple taggants can be read together in a mixed sample and the information encoded in the original taggants separately recovered. Molecular taggants have potential uses in many applications such as tagging items that are not able to be tagged with barcode stickers or radio frequency identification (RFID) tags. Molecular taggants also have advantages over more conventional types of taggants. Molecular taggants can be applied to items that are too small or do not have a suitable place for applying a standard tag. Molecular taggants may be made from edible material and applied to food. However, the use of molecular taggants is not limited to any particular type of item. Molecular taggants may be used in supply chain tracking similar to any other type of taggant. Discussion of molecular taggants and potential usage scenarios can be found in U.S. Pat. Pub. No. 2023/0101409, U.S. Pat. Pub. No. 2023/0125457, U.S. Pat. Pub. No. 2023/0101083, U.S. Pat. Pub. No. 2023/0313276, and U.S. patent application Ser. No. 18/301,455 filed on Apr. 17, 2023.

Molecular taggants may be used to encode a binary barcode. A binary barcode is an arbitrary sequence of bits that, like a conventional barcode, represents some other information. A binary barcode can represent anything that any other barcode could represent such as the name of an item, the price of an item, the source of an item, and the like.

This disclosure relates to the use of encoding to encode a binary barcode with molecular taggants. Encoding schemes that encode binary barcodes could potentially result in bit errors if multiple taggants are mixed. A bit error is a failure to be able to uniquely identify the bits encoded by two or more molecular taggants when those taggants are combined and read together in a single sample. One encoding scheme that could result in bit errors is presence encoding. Another encoding scheme that could result in errors is hybridization encoding.

Presence encoding uses the presence or absence of a particular molecule to represent a bit. One of the two bits (e.g., 1) is represented by the presence of a molecule and the other bit (e.g., 0) is represented by the absence. The choice of presence representing 1 and absence representing 0 is arbitrary and may be switched. One technique for using deoxyribonucleic acid (DNA) strands to encode barcode sequences with presence encoding is described in Doroschack, Kathryn et al., Rapid and robust assembly and decoding of molecular tags with DNA-based nanopore signatures, Nature Comm., 11:5454 (2020). However, other types of molecules besides DNA strands may be used to make molecular taggants. Non-limiting examples of molecules that could be used to make molecular taggant include sequence-defined polymers such as polynucleotides, proteins, or plastics; metals; fluorophores; small organic molecules; and the like including mixtures of different types of molecules.

With presence encoding, a molecular taggant is formed from multiple different molecular bits. Each molecular bit corresponds to a particular species of molecule. The number of different species of molecules used depends on the number of bits in the binary barcode-one molecular species for each bit. The individual molecular bits are typically physically separate from each other and not linked together in a polymer or with any type of covalent or non-covalent attachment.

Consider three-bit digital barcodes. The possible three-bit binary strings are 000, 001, 010, 011, 100, 101, 110, and 111. Molecular taggants representing these binary strings can be encoded by various combinations of three different molecular bits. A first molecular bit represents the first position in the binary string, a second molecular bit represents the middle position, and a third molecular bit represents the third and final position. In an example, the first molecular bit is a green fluorescent molecule, the second molecular bits is a blue fluorescent molecule, and the third molecular bit is a red fluorescent molecule. A typical item tagged with a molecular taggant will be tagged with many copies (hundreds, thousands, or more) of each of the molecular bit. In this example, the barcode 011 is represented by the blue and red molecules. The barcode 110 is represented by the green and blue molecules. Detecting the molecular bit present is a sample taken from the item (using an appropriate detection method for the specific type of molecule) can identify which colors of fluorescent molecules are in the sample and in turn identify the digital barcode.

However, with presence encoding samples that include more than one molecular taggant may give erroneous readings. A sample may have multiple molecular taggants if, for example, it was collected from an item tagged at multiple stages in a supply chain or from a mixed item that contains multiple individually tagged items mixed together. Consider the combination of the molecular taggants representing the digital barcodes 011 and 110. A sample that contains molecular bit from both these molecular taggants will include the green, blue, and red fluorescent molecules. This combination of green, blue, and red, according to the above encoding scheme, represents the digital barcode of 111. Yet that barcode was not applied to any of the items and a readout of 111 fails to recognize that the original barcodes were 011 and 110. This is the potential problem with presence encoding and mixed molecular taggants.

Mathematically, when two presence encoding taggants are mixed in a sample, the result is a combination of the binary barcodes according to a binary “OR” operation. The OR operation is a logical operation that yields true if any one of its conditions is true and false if all conditions are false. Thus, if at a given position for any input barcode the value is 1 (i.e., the corresponding molecular bit is present) then the result of the OR operation is 1 (i.e., that molecular bit will be detected in the sample). For the binary barcodes 011 and 110, the first position is 0/1=1, the second position is 1/1=1, and the third position is 1/1=1, resulting in the binary string 111 as identified above. Thus, decoding the output of a sample that contains a mixture of presence encoding molecular taggants back to the original digital barcodes is not a trivial problem.

A similar problem can exist when hybridization encoding is used to encode a binary barcode with a collection of polynucleotides. Hybridization encoding uses the presence of a second, hybridized polynucleotide strand to encode a bit value. With hybridization encoding, a given polynucleotide strand that exists in a taggant as a single-stranded molecule encodes a first bit (i.e., 0 or 1) and that same polynucleotide strand if present as a double-stranded molecule encodes the other bit. Mixing multiple taggants that use the same polynucleotide strands to encode binary barcodes may result in the creation of a double-stranded molecule as a result of the mixing. This would result in a readout of an incorrect value bit value. Thus, decoding the output of a sample that contains a mixture of hybridization encoding molecular taggants back to the original digital barcodes may be difficult or impossible if there is unintended hybridization.

This disclosure provides a solution for reading mixed taggants created with presence encoding, hybridization encoding, or any other molecular encoding scheme that could lead to bit errors when mixing taggants. The solution as described in detail below is to use a superimposed code when designing the binary barcodes.

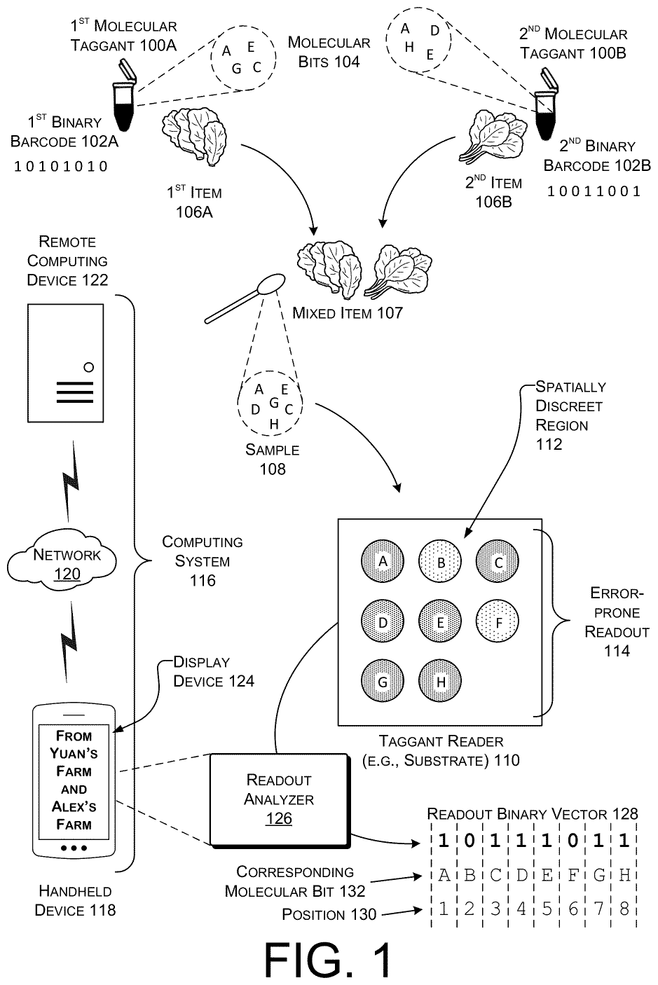

FIG. 1 shows a technique for using molecular taggants 100 in a way that the corresponding binary barcodes 102 can be identified even if the molecular taggants 100 are mixed. In this illustration, a first molecular taggant 100A and a second molecular taggant 100B are each encoded by four molecular bits 104. The individual molecular bits 104 are represented by a capital letter. The molecular bits 104 represent different species of any type of molecule that can be individually detected. For example, the molecular bits 104 could be single or double-stranded polynucleotides in which each molecular bit 104 is represented by a polynucleotide having a different sequence. The first molecular taggant 100A is made up of the molecular bits 104 A, C, E, and G. The second molecular taggant 100B is made up of the molecular bits 104 A, D, E, and H. A molecular taggant 100 may include the molecular bits 104 in a solution (e.g., molecules suspended in water or other fluid) as a dry powder (e.g., a powder containing a mixture of different molecules), or any other format that makes it possible to apply the molecular bits 104 to in item 106.

The first molecular taggant 100A encodes a first binary barcode 102A that may be a binary number of any lengths. This example, it is an eight-bit binary number 10 10 10 10. The second molecular taggant 100B encodes a second binary barcode 102B that is the same length as the first binary barcode 102A. Thus, the second binary barcode 102B is also an eight-bit binary number which, in this example, is 10 01 10 01. The first molecular taggant 100A is applied to a first item 106A. The second molecular taggant 100B is applied to a second item 106B. Although only two items 106 are shown in FIG. 1, this technique may be implemented with a greater number of items.

In this illustration, the first item 106A and the second item 106B are mixed together to create a mixed item 107. For example, the first item 106A and the second item 106B may be lettuce or another type of produce. One use of molecular taggants 100 is for tracing sources of food contamination. For example, molecular taggants 100 may be used to track the source of the lettuce. Thus, the first binary barcode 102A and the second binary barcode 102B may be indicators of the farm that produced the lettuce. Lettuce from different farms may be mixed together to create a bag of salad greens creating the mixed item 107. If someone becomes sick from a bag of salad greens, a barcode on the outside of the bag may not be sufficient to identify the source of the contamination. However, molecular taggants 100 applied to individual pieces of lettuce used to make the bag of salad greens can more precisely identify the source of contamination and aid in identifying the ultimate source of contaminated food.

A sample 108 can be collected from the mixed item 107. The specific technique for collecting the sample 108 will depend on the type of molecule used for the molecular bits 104 and on the type of items 106 in the mixed item 107. In one implementation, a swab may be used to collect material from an outer surface of one or more items within the mixed item 107. The sample 108 should ideally include all the molecular bits 104 present in both the first molecular taggant 100A and in the second molecular taggant 100B. However, it is possible that the sample 108 may fail to include one or more of the molecular bits 104.

This example describes and illustrates the use of multiple molecular taggants 100 on a mixed item 107. However, multiple molecular taggants 100 may also be mixed together when a first molecular taggant 100A and a second molecular taggant are both applied to the same item 106. This may be done as an item 106 travels through different locations in a supply chain. For example, a single item 106 may be initially tagged when it is manufactured, tagged again when it is placed in the warehouse, and tagged a third time when it arrives at a retail location. Thus, a sample 108 that includes molecular bits 104 from multiple molecular taggants 100 may come either from a mixed item 107 or from a single item 106 that has been tagged multiple times.

The sample 108 is read by a taggant reader 110. The taggant reader 110 is a device/system that is used to detect the molecular bits 104 present in the sample 108. The specific design and functionality of the taggant reader 110 will depend upon the type of molecule used for the molecular bits 104. In one implementation, the taggant reader 110 is implemented as a spectrometer. In another implementation, the taggant reader 110 is implemented as a substrate on which there are multiple spatially discrete regions 112 that each detectably changes in the presence of a one of the molecular bits 104. Any number of other implementations are also possible.

One example implementation of a molecular taggant system that allows DNA taggants to be rapidly read on a paper ticket using fluorescence is described in Berk, Kimberly L., et al. “Rapid visual authentication based on DNA strand displacement.” ACS Applied Materials & Interfaces 13.16 (2021): 19476-19486. With this technique a DNA taggant triggers a unique, sequence-driven pattern of fluorescence that one or more spatially discrete regions 112 on a DNA-reporter paper. The presence of a particular DNA molecule (e.g., representing a 1 in a binary barcode 102) is detected by the taggant reader 110 as fluorescence at one of the spatially discrete regions 112. This method is attractive for supply chain tracking due to its low cost and use of only inexpensive equipment. FIG. 1 illustrates this by showing increased fluorescence at the spatially discrete regions 112 corresponding to the molecular bits 104 A, C, D, E, G, and H. The techniques of Burke et al. use a source of ultraviolet light such as an e-gel reader to generate light at the excitation waveband for the fluorophores. A filter is also used so that the light emitted from the fluorophores can be observed by a user or captured with a camera. Thus, the taggant reader 110 may include multiple different components such as an ultraviolet light source, a filter, and a camera or other sensor.

The taggant reader 110 is configured to generate an error-prone readout 114 of the one or more molecular taggants 100 in the sample 108. The readout is referred to as an error-prone readout 114 because it may not accurately report the molecular bits 104 present in the sample 108. As mentioned above, an additional source of error is that the sample 108 may not contain all the molecular bits 104 that were present on the mixed item 107. Contamination or errors in the functioning of the taggant reader 110 may also result in detection of a molecular bit 104 that was not actually present in the sample 108. Thus, the error-prone readout 114 may contain both false positives and false negatives. However, in many cases the error-prone readout will be accurate.

Accordingly, the taggant reader 110 and the error-prone readout 114 are associated with an error rate or error percentage. Work by the inventors using a taggant reader based on the system in Berk et al. resulted in an error rate of about 10%. The specific error rate will vary based on the type of molecule used for the molecular bits 104 and the technology used for the taggant reader 110.

In implementations in which the taggant reader 110 includes a substrate, the error-prone readout 114 is the pattern of detectable changes (e.g., of fluorescence levels) at the spatially discrete regions 112. In other implementations, the error-prone readout 114 may be a chromatogram generated by a mass spectrometer. The specific form of the error-prone readout 114 will of course vary with the type of technology used for the taggant reader 110. The error-prone readout 114 represents the observable results of analyzing the sample 108 with the taggant reader 110.

The error-prone readout 114 is interpreted by a computing system 116 comprising a processing unit and a memory. The computing system 116 may be any type of computing system including one or more computing devices. For example, the computing system 116 may include a handheld device 118. The handheld device 118 may be a smart phone or any other type of handheld device that can function as a computing device. In some implementations, the handheld device 118 may also function as a sensor such as by using a camera to capture an image of a substrate that shows the error-prone readout 114. The error-prone readout 114 may also be captured by a sensor that is not a component of the computing system 116.

In some implementations, the handheld device 118 in the computing system 116 is connected via a network 120 to a remote computing device 122. The network 120 may be any type of communication such as the Internet. The remote computing device 122 may be any type of computing device that is located at a physical distance and is remote with respect to the taggant reader 110. Examples of a remote computing device 122 include a network-accessible computer or group of computers such as “cloud” implementation. The computing system 116 also includes one or more output devices such as a display device 124. The display device 124 may be component of handheld device 118 or it may be a part of a different portion of the computing system 116. The computing system 116 may cause the display device 124, or other output device, to render a human-readable output representing the information encoded by the first binary barcode 102A and the second binary barcode 102B.

The computing system 116 includes a readout analyzer 126. The readout analyzer 126 is configured to convert the error-prone readout 114 generated by that taggant reader 110 to a readout binary vector 128. The readout analyzer 126 may be implemented as software code, firmware, or combination thereof. The readout analyzer 126 may be implemented by any of the components of the computing system 116 or in part by multiple different components. If the taggant reader 110 is implemented as a substrate, the readout analyzer 126 may compare before and after levels for each of the spatially discrete regions 112 to determine which regions observably react (e.g., fluoresce) after contact with the sample 108. This observable reaction at one of the spatially discrete regions 112 is then interpreted by the readout analyzer 126 as indicating presence of the corresponding molecular bit 104.

The readout binary vector 128 is a binary string of the same length as the first binary barcode 102A and the second binary barcode 102B. Each position 130 in the readout binary vector 128 is associated with a corresponding molecular bit 132. In this example, the readout binary vector 128 is an eight-bit vector that includes eight positions numbered from left to right. The ordering of the positions 130 is arbitrary. They may also be ordered from right to left. Each position in the readout binary vector 128 corresponds to one of the possible molecular bits 104 that may be used to create a binary barcode 102. In this example, there are eight possible molecular bits 104 represented by the capital letters A-H. The error-prone readout 114 (which in this example is error free) detects the presence of molecular bits 104 A, C, D, E, G, and H. This is represented by the readout binary vector 128 having a 1 at the first, third, fourth, fifth, seventh, and eighth position 130.

This readout binary vector 128 also represents (in the absence of error) the binary OR operation on the first binary barcode 102A and the second binary barcode 102B. Thus, in this example, the readout binary vector 128 of 10111011 is the result of taking the binary OR operation on 10101010 and 10011001. However, due to the possible presence of errors, the readout binary vector 128 is a noisy version of the purely mathematical OR operation on the first binary barcode 102A and the second binary barcode 102B. If there is error, the readout binary vector 128 may not accurately represent the results of the binary OR operation on the first binary barcode 102A and the second binary barcode 102B. However, without a way to assign meaning to the readout binary vector 128 in the presence of error, is not possible for the computing system 116 to identify the first binary barcode 102A and the second binary barcode 102B from the sample 108.

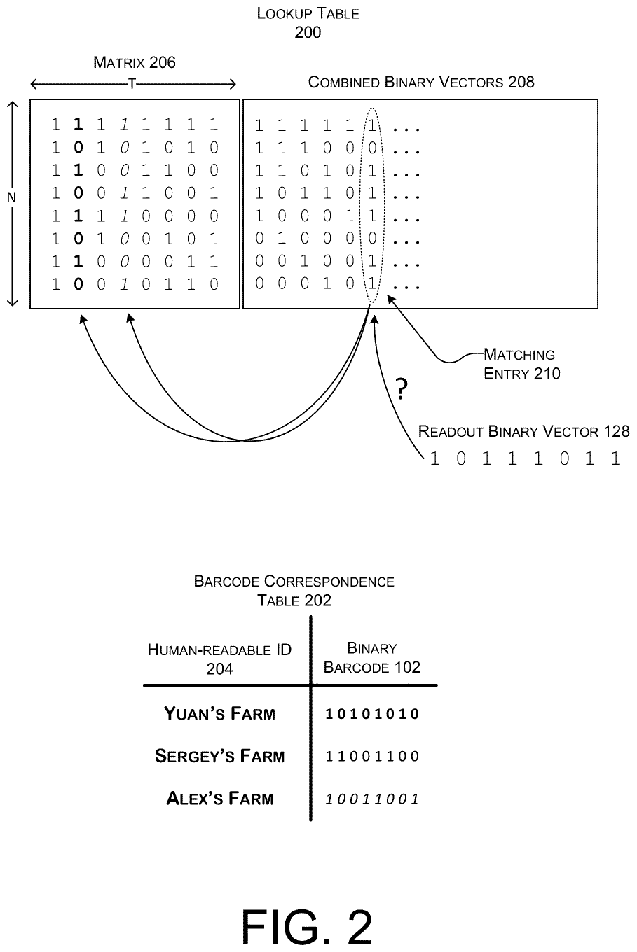

FIG. 2 provides illustrative examples of two types of tables that may be used by the computing system 116 to interpret molecular taggants 100 as represented by the binary vector 128. The first table is a lookup table 200 in which the readout binary vector 128 is used to query the lookup table 200 and identify one or more binary barcodes 102. The second table is a barcode correspondence table 202 used to identify a human-readable identifier 204 associated with a binary barcode 102.

The solution to the problem that can arise from combining molecular taggants 100 which use presence encoding is to use a superimposed code when designing the binary barcodes 102. A superimposed code limits the possible binary barcodes 102 that can be used. The set of valid binary barcodes is restricted to those that have certain properties, specifically those in which the resulting readout binary vector 128 can be decoded back into the original binary barcodes 102 even when multiple molecular taggants 100 are combined.

The goal of using a superimposed code is to create a collection of T binary strings (i.e., possible binary barcodes 102) of length N so that the bitwise OR of at most S of them results in a unique binary string. The number of valid binary barcodes according to the superimposed code is thus T. The number of bits in each of the valid binary barcodes is thus N (N=8 in the example shown in FIG. 1). S is a parameter that bounds how many different binary barcodes 102 can be recovered from a readout binary vector 128. For example, if S=2 then two different binary barcodes 102 can be mixed and accurately read out from a sample 108. Thus, the lookup table 200 is generated based on a superimposed encoding and comprises a plurality of binary vectors that each uniquely correlate to one or more binary barcodes.

One way to represent the set of valid binary barcodes is in a N×T matrix 206 with each column in the matrix 206 representing a different valid binary barcode. A dimension of the matrix 206, N and/or T, is the same as a number of positions in the binary barcode 102. The matrix 206 may exclude the all-zero vector because that would be represented by the complete absence of any molecular bit 104. It is to be understood that the distinction between a column and row in the matrix 206 is arbitrary and discussions of a column could apply equally to a row simply by rotating the matrix 206. The term “entry” is used to refer to either a column or row of the matrix 206. In the matrix 206, the binary vector in each column is different from the binary vector in every other column. The matrix 206 also has the property that the bitwise OR of any S (e.g., 2, 3, etc.) binary vectors from the matrix 206 generates another binary vector that is (i) distinct from all binary vectors in the matrix 206 and (ii) distinct from all other binary vectors generated from the bitwise OR of any other S binary vectors from the matrix 206. These binary vectors generated by using the bitwise OR operation on S columns from the matrix 206 referred to herein as “combined binary vectors” 208. Using the notation where the matrix 206 includes T columns each containing a binary vector and S is the number of “combinable” binary vectors, the concept of combining every S binary vectors from the matrix 206 can be represented in combinatorial language as T choose S.

Thus, lookup table 200 may be implemented as collection of binary vectors having certain properties. This collection of binary vectors includes the matrix 206 and may, but need not, include the combined binary vectors 208 generated by performing the bitwise OR operation on T choose S binary vectors from the matrix 206. The combined binary vectors 208 may be thought of as a second matrix concatenated to the matrix 206 that can be available as a lookup table in some implementations. The collection of combined binary vectors 208 may make a matrix that is much larger than the matrix 206 from which they were generated. If each binary vector of the combined binary vectors 208 is to resolve uniquely to two different entries in the matrix 206 (i.e., S=2) then the number of columns in the matrix containing the combined binary vectors 208 can be calculated by [T×(T−1)]/2. If, as an example shown FIG. 2, T=8 then there are [8×7]/2=28 unique combined binary vectors 208. For the sake of simplicity, only six combined binary vectors 208 are illustrated in FIG. 2. Combining this with the matrix 206 results in an 8×28 matrix being appended to an 8×8 matrix to create a lookup table 200 that contains 36 eight-bit entries. However, for the purposes of performing a lookup, the specific format, shape, vector order, and other features of the lookup table 200 may be freely changed so long as every entry in the lookup table 200 retains the specified properties. Moreover, the combined binary vectors 208 may be generated from the vectors in the matrix 206 during a lookup operation, and thus, there is no need for these vectors to be precomputed or stored in a separate table.

In order to accommodate the presence of two-way errors in the error-prone readout 114, lookup table 200 can also include an additional property. Specifically, every column in the lookup table 200 is sufficiently dissimilar from every other column. This makes it possible to uniquely match the readout binary vector 128 to one of the columns in the lookup table 200 with the presence of errors. The concept of “sufficiently dissimilar” may be represented as two binary vectors differing by least a threshold Hamming distance. The Hamming distance is a metric for comparing two binary data strings of equal length. It is the number of bit positions in which the corresponding bits are different. This may be represented as a percentage by dividing the number of bit positions that are different by the total number of bits in the binary vector. For example, if two bits are different between two 8-bit binary vectors the Hamming distance is 2 which may also be represented as a difference of 25%.

The lookup table 200 may be designed so that the Hamming distance between every two binary vectors in the lookup table 200 is at least twice the error percentage of the error-prone readout 114. The error percentage of the error-prone readout 114 may be determined experimentally for a given system or a likely error percentage may be inferred or calculated. For example, if the error-prone readout 114 has an error rate of about 10% then the Hamming distance between any two binary vectors in the lookup table would be at least about 20%. Similarly, if the lookup table 200 is designed so that the Hamming distance between any two binary vectors is 25%, then the system could accommodate up to a 12.5% error rate in the error-prone readout 114. The threshold Hamming distance may apply to all binary vectors the lookup table 200 including those in the matrix 206 as well as the combined binary vectors 208.

The computing system 116 may use the lookup table 200 by comparing the readout binary vector 128 to each entry in the lookup table 200 and identifying a matching entry 210. This may be implemented by instructions stored in memory of the computing system 116 that are executable on a processing unit which cause the computing system 116 to compare the readout binary vector 128 to the lookup table 200 and identify the matching entry 210. The matching entry 210 is the entry in the lookup table 200 that is the most similar to the readout binary vector 128. In many instances, there will be a binary vector in the matrix 206 that exactly matches the readout binary vector 128. However, due to the presence of errors there may also be instances in which the readout binary vector 128 does not exactly match any entry in the lookup table 200. The matching entry 210 is the entry in the lookup table 200 that has the smallest Hamming distance from the readout binary vector 128. However, other metrics for comparing vectors besides Hamming distance may be used such as cosine similarity.

By setting the Hamming distance between all of the binary vectors in the lookup table 200 to at least twice the error rate of the error-prone readout 114 it makes it possible to unambiguously map the readout binary vector 128 to a single matching entry 210. If the difference between any two binary vectors in the lookup table 200 is more than twice the likely error, it is unlikely that the readout binary vector 128 will be equally similar (i.e., “right in the middle”) to two different entries. The error rate of the error-prone readout 114 will in most implementations be an average error rate for the system. Thus, it is possible in practice there may be a readout binary vector 128 with a higher level of error that cannot be resolved unambiguously to a single entry in the lookup table 200. However, the probability of this happening is greatly reduced by making the distance between each entry in the lookup table 200 at least twice the expected error rate.

If the readout binary vector 128 is matched to one of the binary vectors in the matrix 206, then that vector from the matrix 206 is the binary barcode 102. This is the expected result when only a single binary barcode 102 is contained in the sample 108. When the task is to identify only a single binary barcode 102 number of entries to be found in the matrix 206 is 1 which can be represented as S=1. One illustrative way to find the best match from matrix 206 is to evaluate every column from matrix 206 and identify the current best match (1) and the number of bits (2) that matched in the best match. Recall that because of errors, the readout binary vector 128 may not exactly match any of the columns in the matrix 206. Each column in the matrix 206 is considered and evaluated to determine if it has more matches with the readout binary vector 128 than the current best match (1). If it does, (1) is updated to the current column and (2) is updated to the number of matches the current column has with the readout binary vector 128. Once all columns of the matrix 206 are considered, (1) will store the best match overall.

If there are molecular bits 104 representing multiple binary barcodes 102 in the sample 108, then the readout binary vector 128 will match one of the combined binary vectors 208. A combined binary vector 208 is used to identify the S entries in the matrix 206 from which it was generated by the binary OR operation. FIG. 2 illustrates an example when S=2 and the matching entry 210 corresponds two rows of the matrix 206. These two rows represent two binary barcodes 102, and thus, two mixed molecular taggants 100 are both uniquely identified from a mixed sample 108. If S is a larger number than two, a matching entry 210 from combined binary vectors 208 would correlate with a larger number of columns, S columns, in the matrix 206.

When S=2, or any value greater than 1, the combined binary vectors 208 can be computed on the fly without being previously stored. The same operations may be performed as described above in the case when S=1. However, for every column in the matrix 206 the bitwise OR of that column with every combination of up to S−1 other columns (i.e., all sets of 2, 3, . . . , S columns that include the current column) is generated. For each set of columns, the bitwise OR is compared to the current best match, and if better, the set of columns used in the bitwise OR (1) is stored and (2) the updated count of matching bits. Once all columns in the matrix 206 are evaluated in this way, (1) will store the best matching S columns from the matrix 206.

Storing the entire set of combined binary vectors 208 in memory can increase the speed of lookup but requires more memory (e.g., RAM) and disk space. The number of combined binary vectors 208 can become large very quickly as the length of the vectors increase and as S increases. In an implementation, consistent ordering of the combined entries can be relied upon to identify the input entries. For example, if there are 3 columns in the matrix 206 there will necessarily be the following 6 columns in the lookup table 200 in the following order for S=2: [1, 2, 3, 1 OR 2, 1 OR 3, 2 OR 3] (the same relationships hold for S>2). When a lookup is performed, the input is compared against all columns in the lookup table 200 and the index of the best match (1) as well as the number of bits that matched in the best match (2) are both stored. This is the same process as in the S=1 case but modified to store the INDEX of the best match (1). After evaluating the entire lookup table 200, the index of the best matching column uniquely identifies S vectors in the matrix 206.

Any possible technique for creating a lookup table 200 with the desired properties may be used. In some implementations, a matrix 206 with the desired properties is used to generate the lookup table 200. There are multiple types of matrices that arise from combinatorial design theory such as, but not limited to, Latin squares, incidence matrices of expander graphs, balanced incomplete block designs, difference sets, and Hadamard matrices that can be used to provided different quantitative parameters suitable for designing binary barcode. In one specific implementation the matrix 206 is a Hadamard matrix. A Hadamard matrix is a square, binary matrix whose entries are either 1 or 0. The dimension of a Hadamard matrix is 2^{x-1}×2^{x-1} where x is any arbitrary integer. Additionally, the columns of a Hadamard matrix are mutually orthogonal. Thus, each pair of columns in a Hadamard matrix represents two perpendicular binary vectors, while in combinatorial terms, it means that each pair of columns has matching entries in exactly half of their rows and mismatched entries in the remaining rows. It is a consequence of this definition that the corresponding properties hold for rows as well as columns. Hadamard matrices also have the property that the bitwise OR operation performed on any two entries generates a combined binary vector 208 that is different from all the binary vectors in the Hadamard matrix 206 as well as all other combined binary vectors 208.

Hadamard matrices have another desirable property. Each entry has a Hamming distance of at least 50% from every other entry. This level of dissimilarity for the combined binary vectors 208 created by the binary OR operation depends on the number of entries that can be combined. If S=2 and the combined binary vectors 208 represent combinations of any two entries from a Hadamard matrix, the minimum Hamming distance will be 25%. The minimum Hamming distance will decrease as the number of potentially combinable entries, the value of S, increases. The minimum Hamming distance is 50/S so it will be 25% when S=2, 16.7% when S=3, etc.

There are multiple possible techniques that can be used to generate a Hadamard matrix. Any is suitable. One of the most common methods is Sylvester's construction which generates a Sylvester-type Hadamard matrix. This method involves constructing a Hadamard matrix of order 2ⁿ from a Hadamard matrix of order n by partitioning the matrix into four quadrants and filling them with the original matrix and its negation. Sylvester's matrices have a number of special properties. They are symmetric and, when k≥1 (2 k>1), have trace zero. The elements in the first column and the first row are all 1. The elements in all the other rows and columns are evenly divided between 1 and 0.

Another method for generating Hadamard matrices is the Paley construction, which can be used to construct Hadamard matrices when the order is divisible by 4 and of the form q^{k}×4^{l}, where q is an odd prime, k is a positive integer, and l is a nonnegative integer 1.

Accordingly, the matrix 206 may be a Hadamard matrix and the set of valid binary barcodes are the columns (or rows) of the Hadamard matrix. Because it is a square matrix, the length of each binary barcode 102 will be equal to the number of valid binary barcodes for a given encoding scheme. In the example matrix 206 shown in FIG. 2, there are eight, 8-bit valid binary barcodes.

Once one or more binary barcodes 102 are identified from the lookup table 200, those binary barcodes 102 may be correlated with human-readable identifiers 204. The human-readable identifiers 204 represent any type of label or information that is intended to be associated with an item 106 through the application of a molecular taggant 100. This can be any type of information typically represented by a conventional barcode. For example, it may be the name of farm that produced a head of lettuce. There are multiple possible ways to correlate a binary barcode 102 with a human-readable identifier 204 and any suitable technique may be used. In one implementation, a barcode correspondence table 202 has entries that record correspondence between a particular binary barcode 102 and the human-readable identifier 204 represented by that barcode. Once the human-readable identifier(s) 204 are determined, they may be displayed on the display device 124 or presented to a user in another matter.

It is not, however, necessary in all implementations that the binary barcodes 102 correlate with human readable identifiers 204. In some implementations, the binary barcodes 102 may be used as binary strings for downstream processes or analyses by the computing system 116 and there may not be any corresponding human-readable identifiers 204.

### Illustrative Methods

For ease of understanding, the processes discussed in this disclosure are delineated as separate operations represented as independent blocks. However, these separately delineated operations should not be construed as necessarily order-dependent in their performance. The order in which the processes are described is not intended to be construed as a limitation, and unless other otherwise contradicted by context any number of the described process blocks may be combined in any order to implement the process or an alternate process. Moreover, it is also possible that one or more of the provided operations is modified or omitted.

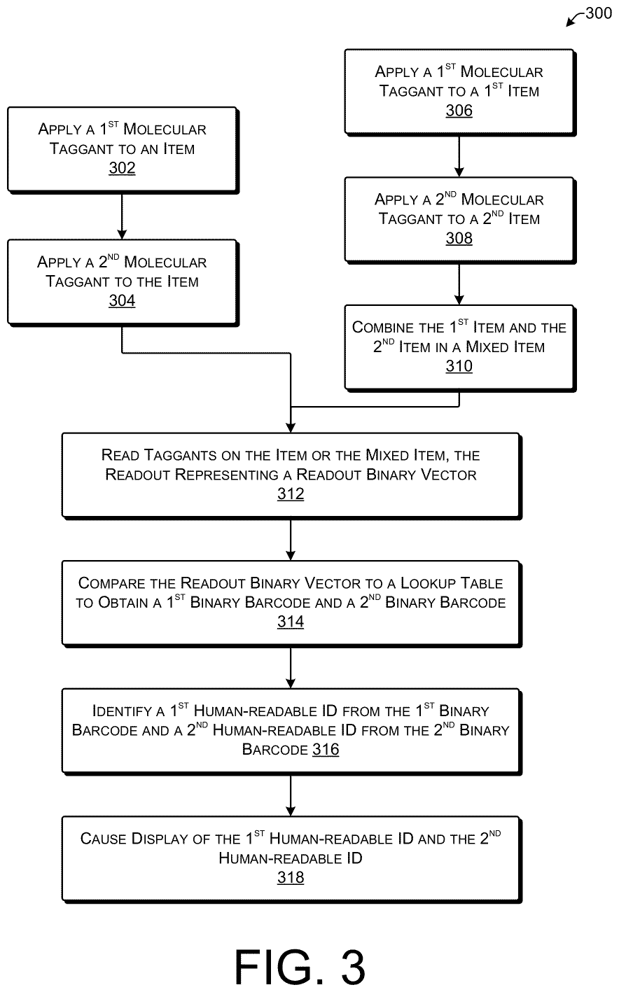

FIG. 3 shows an illustrative method 300 for applying molecular taggants to one or more items, reading the molecular taggants, and displaying human-readable identifiers corresponding to the taggants. Method 300 may be implemented by the systems and tables shown in FIGS. 1 and 2. Method 300 may begin one of two ways: with multiple molecular taggants applied to the same item or with molecular taggants applied to different items that ultimately combined into a mixed item.

At operation 302, a first molecular taggant is applied to an item. The molecular taggant uses a presence encoding scheme comprising molecular bits to encode a first binary barcode. In implementation, the first molecular taggant is applied to the item at a first location in supply chain. For example, the first location may be a factory where the item is manufactured or farm where the item is grown.

At operation 304, a second molecular taggant is applied to the item. The second molecular taggant uses the same presence encoding scheme as the first molecular taggant. The second molecular taggant uses the molecular bits to encode a second binary barcode. By way of explanation, when using the same presence encoding scheme, the presence or absence of the same molecule will represent the first molecular bits in both the binary barcode encoded by the first molecular taggant in the binary barcode encoded by the second molecular taggant. Thus, at this point a single item is tagged with molecular bits corresponding to two different molecular taggants. The second molecular taggant may be applied at a second location in a supply chain. For example, the second location may be a warehouse or processing center that the item passes through in route to the end consumer.

At operation 306, a first molecular taggant that uses a presence encoding scheme comprising molecular bits to encode a first binary barcode is applied to the first item that will be included in a mixed item. This operation is analogous to operation 302 except that the molecular taggant is applied to an item that will be combined with another item to make a mixed item. The first item could be any type of item that is combined with other items to make a mixed item. For example, the first item could be lettuce or another type of foodstuff that is combined with other foods for example in a bag of salad mix.

At operation 308, a second molecular taggant that uses the same presence encoding scheme comprising molecular bits to encode a second binary barcode is applied to a second item that will also be included in the mixed item. The second item can be the same or different type of item as the first item. For example, both the first and second items could be diamonds of the same size, quality, and cut combined together in a piece of jewelry that is the mixed item.

At operation 310, the first item and the second item are combined in a mixed item. For example, lettuce from the first farmer and lettuce from a second farmer may be combined in a bag of salad mix. Similarly, a diamond from a first source may direct from a second source may be combined into a piece of jewelry. Once combined into a mixed item, a sample may be collected from the mixed item that will contain the molecular bits of the first molecular taggant and the second molecular taggant.

The first molecular taggant and the second molecular taggant use the same presence encoding scheme, and thus, are based on selections from the same set of molecular bits. Accordingly, the first molecular taggant and the second molecular taggant comprise one or more species of whatever type of molecule is used for the molecular bits. For example, the first molecular taggant and the second molecular taggant may both comprise a plurality of sequence-defined polymers in which each unique sequence-defined polymer is associated with a single molecular bit. The sequence-defined polymers may be polynucleotides (e.g., DNA or RNA), proteins, plastics polymers, or another type of polymer with a detectable sequence of at least two different monomer units.

One advantage of using a collection of molecular bits as a molecular taggant is lower cost. Molecular taggants could alternatively be made from sequence-defined polymers in which each molecular taggant is a single polymer with a unique sequence. This, however, requires synthesizing a custom polymer for each item that is tagged. If, for example, DNA is used to create molecular bits, many copies of a few different DNA sequences can be synthesized in bulk. This results in lower manufacturing costs because once a single DNA sequence is synthesized using standard synthesis techniques, many additional copies can be made rapidly and at low cost through techniques such as polymerase chain reaction (PCR) or by creating multiple copies using an organism such as E. coli. It is much less expensive to create a molecular taggant from the combination of multiple inexpensively-produced DNA strands than to perform de novo synthesis and create a unique DNA strand for tagging each item.

As described above, the first binary barcode and the second binary barcode may both have superimposed encoding. Thus, the first binary barcode and the second binary barcode may both be binary vectors from the matrix. The binary vectors are entries in the matrix meaning they are either columns or rows of the matrix. The matrix may be a square, binary matrix. In some implementations, the matrix is a Hadamard matrix.

At operation 312, molecular taggants on the item (from operations 302 and 304) or on the mixed item (from operation 310) are read. The molecular taggants are read from a sample from the item or the mixed item. The sample may be obtained in a number of ways depending on the type of the item and the type of molecule used for the molecular taggants. For example, a portion of the item may be swabbed and the swab may be soaked in a solution to obtain a solution containing the molecular bits. Alternatively, the item may be rinsed with a solution so that molecular bits on the surface of the item and to the solution and the sample is taken from this solution.

Reading the molecular taggants generates an error-prone readout. Errors arise both from contamination or damage to the molecular taggants on the item and from errors introduced by a taggant reader that reads the molecular taggants. Due to the presence encoding scheme in which the presence or absence of specific molecular bits encodes either a 0 or 1, the error-prone readout represents a readout binary vector. Although the value of the readout binary vector is generated by a taggant reader detecting the presence or absence of specific molecular bits in a sample, the value of that readout binary vector (absent error) represents a vector generated by a bitwise OR operation on the first binary barcode and the second binary barcode.

In one implementation, reading molecular taggants is performed by contacting a substrate with the molecular bits from the first molecular taggant the second molecular taggant. The substrate may be a piece of paper such as nitrocellulose paper that is configured to bind DNA. The molecular bits are brought in contact with the substrate by first collecting a sample of the molecular taggants from the item or mixed item and applying the sample to the substrate. In this implementation, the substrate is configured to have a plurality of spatially discrete regions that each observably reacts to an individual one of the molecular bits. For example, the substrate may have one region for each bit in the binary barcodes and each of those regions may hybridize to a DNA molecule of a specific sequence (i.e., one of the molecular bits) thereby fluorescence that can be visibly detected.

At operation 314, the readout binary vector compared to a lookup table. The lookup table may comprise a matrix such as a Hadamard matrix. The lookup table is generated based on a superimposed encoding and is configured to correlate the readout binary vector to the binary barcodes present in the sample. In this example method 300, lookup table would correlate the readout binary vector with the first binary barcode and the second binary barcode. The superimposed encoding creates a relationship between all valid binary barcodes (of which the first binary barcode and the second binary barcode represent two) such that the bitwise OR operation performed on any N valid binary barcodes generates a combined binary vector that is different from all the valid binary barcodes and from all other combined binary vectors.

The lookup table may have additional property that each entry has at least a threshold Hamming distance from every other entry in the lookup table. Having sufficient difference between each entry in the lookup table makes it possible in most cases to match the readout binary vector with a single entry even if there is no exact match because of errors present in the readout binary vector. In some implementations, the threshold Hamming distance is at least twice an error rate of the error-prone readout.

At operation 316, a first human-readable identifier is identified from the first binary barcode and a second human-readable identifier is identified the second binary barcode. This represents decoding the binary barcodes into information that is meaningful to a human user. In some implementations, the binary barcodes are correlated with human-readable identifiers to use of a barcode correspondence table.

At operation 318, method 300 includes causing display of the first human-readable identifier and the second human-readable identifier. The human-readable identifiers may be displayed on a display device such as that of a handheld device operated by a user who is reading the first molecular taggant and the second molecular taggant. Thus, in this way the user is able to conveniently access information encoded by the molecular taggants while inspecting the item or mixed item.

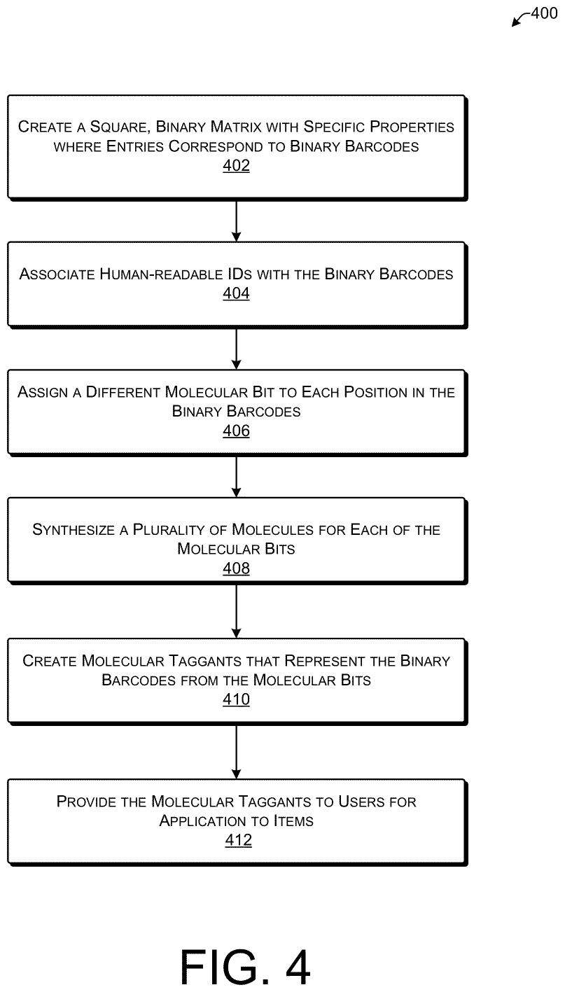

FIG. 4 shows an illustrative method 400 for designing and creating molecular taggants that can be read and decoded when mixed. Method 400 may be implemented by the systems and tables shown in FIGS. 1 and 2.

At operation 402, a square, binary matrix is created with specific properties. This matrix has the property that every entry in the matrix is different and that a bitwise OR operation on any two entries in the matrix creates a combined binary vector that is different from each entry in the matrix and from every other combined binary vector created by the bitwise OR operation on any other two entries in the matrix. The matrix may be a Hadamard matrix. Each entry in the matrix can represent either a column or row of the matrix. In the matrix, at least one entry corresponds to a binary barcode. That is, the bit series that makes of a row or column of the matrix is the same series of 0s and 1s in a binary barcode. In some implementations, every entry in the matrix corresponds to a binary barcode.

The matrix may also have the property such that every entry in the matrix and every combined binary vector differs by at least a threshold Hamming distance. The threshold Hamming distance may be at least twice the expected error for a system that reads the molecular taggants. The entries in the matrix may comprise the set of valid binary barcodes for a given presence encoding scheme.

At operation 404, a human-readable identifier is associated with the binary barcode. The human-readable identifier may be associated with the binary barcode by the use of a barcode correspondence table. The set of valid binary barcodes represents barcodes that could potentially be used but in some implementations there may not be a human-readable identifier associated with every barcode. There may be unused barcodes.

At operation 406, a different molecular bit is assigned to each position in the binary barcodes. Each molecular bit is a molecule that can be differentiated from all other molecular bits used in a given presence encoding scheme. For example, the molecule could be a sequence-defined polymer and each molecular bit is differentiated from all the other molecular bits based on the respective polymer sequences. In an implementation in which the molecular bits are DNA strands, the molecular bit corresponding to the first bit position in the binary barcodes will be a DNA strand with a first sequence, the molecular bit corresponding to the second bit position the binary barcodes will be a DNA strand with a second sequence, and so forth. In this example, all the DNA strands can be differentiated from each other based on their nucleotide sequences.

At operation 408, a plurality of molecules are synthesized for each of the molecular bits representing each position in the binary barcodes. Thus, if the binary barcode is a 32-bit barcode there would be 32 different molecules synthesized. Even though no molecular taggant will by itself use all of these molecules, all 32 different molecules may be needed to create multiple different molecular taggants. Large quantities of each of the molecules may be pre-synthesized in bulk by efficient and cost-effective techniques. For example, multiple pools of molecular bits could be created-one pool for each type of molecular bit used in the presence encoding scheme. Continuing with the prior example, there could be 32 different pools each containing a large number of molecules used to encode 32-bit binary barcodes. For example, all 32 molecules can be pre-synthesized and stored in separate pools labeled with their position in the taggant vector. When creating a molecular taggant, samples are gathered from each pool where the taggant vector value is 1 (assuming presence is used to encode the bit value 1; presence could alternatively be used to encode 0). This subset of samples is mixed to create the 32-bit molecular taggant.

At operation 410, a molecular taggant is created that represents the binary barcode according to the presence encoding scheme. The molecular taggant includes the molecular bit that corresponds to each position in the binary barcode that has a first bit (e.g., 1) and omits the molecular bit corresponding to each position in the binary barcode that has a second bit (e.g., 0). The correspondence between 1 and presence of a molecular bit with 0 been represented by the absence of molecular bit is arbitrary. Alternatively, 0 could be represented by the presence of molecular bit and 1 represented by its absence.

In some implementations, the molecular taggant can be created by taking molecular bits corresponding to each position in the binary barcode that has the first bit (i.e., the digit encoded by presence of a molecule) from pools of pre-synthesized molecular bits and combining those molecular bits. The molecular taggant may comprise different numbers of molecules for each of the molecular bits present in the taggant. For example, a million molecules may be taken from a first pool representing the third bit position in the binary barcode while 1.2 million molecules are taken from a different pool that represents the fifth bit position in the binary barcode and combined with varying numbers of molecules from other pools to create the molecular taggant.

At operation 412, the molecular taggant may be provided to a user for the purpose of applying to an item. Once designed and synthesized, a producer of the molecular taggant may combine the molecular bits together to encode a particular binary barcode and provide the taggant itself (e.g., a liquid containing the molecular bits) and the corresponding binary barcode to a user.

### Illustrative Computer Architecture

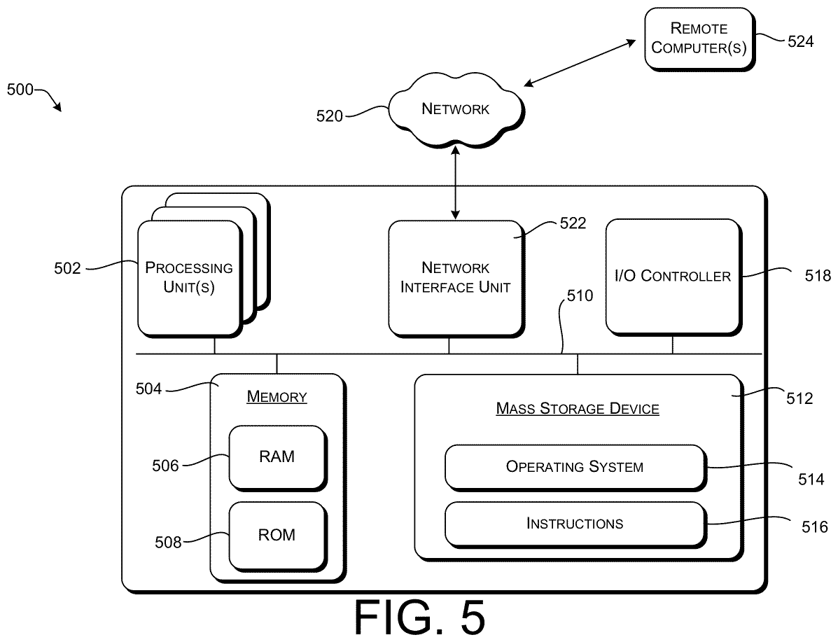

FIG. 5 is a computer architecture diagram showing an illustrative computer hardware and software architecture for a computing device and system such as the computing system 116 introduced FIG. 1. In particular, the computer 500 illustrated in FIG. 5 can be utilized to receive the error-prone readout 114 and/or to maintain a lookup table 200 as shown in FIGS. 1 and 2.

The computer 500 includes one or more processing units 502, a system memory 504, including a random-access memory 506 (“RAM”) and a read-only memory (“ROM”) 508, and a system bus 510 that couples the memory 504 to the processing unit(s) 502. A basic input/output system (“BIOS” or “firmware”) containing the basic routines that help to transfer information between elements within the computer 500, such as during startup, can be stored in the ROM 508. The computer 500 further includes a mass storage device 512 (which also functions as a memory) for storing an operating system 514 and other instructions 516 that represent application programs and/or other types of programs. The mass storage device 512 can also be configured to store files, documents, and data. In some implementations, readout analyzer 126, lookup table 200, and/or the barcode correspondence table 202 may be maintained in the mass storage device 512.

The mass storage device 512 is connected to the processing unit(s) 502 through a mass storage controller (not shown) connected to the bus 510. The mass storage device 512 and its associated computer-readable media provide non-volatile storage for the computer 500. Although the description of computer-readable media contained herein refers to a mass storage device, such as a hard disk, CD-ROM drive, DVD-ROM drive, or USB storage key, it should be appreciated by those skilled in the art that computer-readable media can be any available computer-readable storage media or communication media that can be accessed by the computer 500.

Communication media includes computer-readable instructions, data structures, program modules, or other data in a modulated data signal such as a carrier wave or other transport mechanism and includes any delivery media. The term “modulated data signal” means a signal that has one or more of its characteristics changed or set in a manner so as to encode information in the signal. By way of example, and not limitation, communication media includes wired media such as a wired network or direct-wired connection, and wireless media such as acoustic, radio frequency, infrared, and other wireless media. Combinations of any of the above should also be included within the scope of computer-readable media.

By way of example, and not limitation, computer-readable storage media can include volatile and non-volatile, removable and non-removable media implemented in any method or technology for storage of information such as computer-readable instructions, data structures, program modules, or other data. For example, computer-readable storage media includes, but is not limited to, RAM 506, ROM 508, EPROM, EEPROM, flash memory or other solid-state memory technology, CD-ROM, digital versatile disks (“DVD”), HD-DVD, BLU-RAY, 4K Ultra BLU-RAY, or other optical storage, magnetic cassettes, magnetic tape, magnetic disk storage or other magnetic storage devices, or any other medium that can be used to store the desired information and which can be accessed by the computer 500. For purposes of the claims, the phrase “computer-readable storage medium,” and variations thereof, does not include waves or signals per se or communication media.

According to various configurations, the computer 500 can operate in a networked environment using logical connections to a remote computer(s) 524 through a network 520. The network 520 may be the same as the network 120 shown in FIG. 1. For example, if the computer 500 corresponds to the handheld device 118 then the remote computer 524 may correspond to the remote computing device 122. Alternatively, the computer 500 may correspond to the remote computing device 122 and the remote computer 524 may correspond to the handheld device 118. The computer 500 can connect to the network 520 through a network interface unit 522 connected to the bus 510. It should be appreciated that the network interface unit 522 can also be utilized to connect to other types of networks and remote computer systems. The computer 500 can also include an input/output controller 518 for receiving and processing input from a number of other devices, including a keyboard, mouse, touch input, camera, and an electronic stylus (not shown). Similarly, the input/output controller 518 can provide output to a display screen such as the display device 124 from FIG. 1 or other type of output device (not shown).

It should be appreciated that the software components described herein, when loaded into the processing unit(s) 502 and executed, can transform the processing unit(s) 502 and the overall computer 500 from a general-purpose computing device into a special-purpose computing device customized to facilitate the functionality presented herein. The processing unit(s) 502 can be constructed from any number of transistors or other discrete circuit elements, which can individually or collectively assume any number of states. More specifically, the processing unit(s) 502 can operate as a finite-state machine, in response to executable instructions contained within the software modules disclosed herein. These computer-executable instructions can transform the processing unit(s) 502 by specifying how the processing unit(s) 502 transitions between states, thereby transforming the transistors or other discrete hardware elements constituting the processing unit(s) 502.

Encoding software modules can also transform the physical structure of the computer-readable media presented herein. The specific transformation of physical structure depends on various factors, in different implementations of this description. Examples of such factors include, but are not limited to, the technology used to implement the computer-readable media, whether the computer-readable media is characterized as primary or secondary storage, and the like. For example, if the computer-readable media is implemented as semiconductor-based memory, the software disclosed herein can be encoded on the computer-readable media by transforming the physical state of the semiconductor memory. For instance, the software can transform the state of transistors, capacitors, or other discrete circuit elements constituting the semiconductor memory. The software can also transform the physical state of such components to store data thereupon.

As another example, the computer-readable media disclosed herein can be implemented using magnetic or optical technology. In such implementations, the software presented herein can transform the physical state of magnetic or optical media, when the software is encoded therein. These transformations can include altering the magnetic characteristics of particular locations within given magnetic media. These transformations can also include altering the physical features or characteristics of particular locations within given optical media, to change the optical characteristics of those locations. Other transformations of physical media are possible without departing from the scope and spirit of the present description, with the foregoing examples provided only to facilitate this discussion.

In light of the above, it should be appreciated that many types of physical transformations take place in the computer 500 to store and execute software components and functionalities presented herein. It also should be appreciated that the architecture shown in FIG. 5 for the computer 500, or a similar architecture, can be utilized to implement many types of computing devices such as desktop computers, notebook computers, servers, supercomputers, gaming devices, tablet computers, and other types of computing devices known to those skilled in the art. It is also contemplated that the computer 500 might not include all of the components shown in FIG. 5, can include other components that are not explicitly shown in FIG. 5, or can utilize an architecture completely different than that shown in FIG. 5.

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A method of tagging and reading taggants on items comprising: applying a first molecular taggant (100A) that uses an encoding scheme comprising molecular bits (104) to encode a first binary barcode (102A) to (i) an item (106) or to (ii) a first item (106A) that will be included in a mixed item (107); applying a second molecular taggant (100B) that uses the encoding scheme comprising the molecular bits (104) to encode a second binary barcode (102B) to (i) the item (106) or to (ii) a second item (106B) that will be included in the mixed item (107); reading molecular taggants (100A, 100B) in a sample (108) from (i) the item or the (ii) mixed item thereby generating an error-prone readout (114) (e.g., pattern of fluorescence on a ticket), wherein due to the presence encoding scheme the error-prone readout (114) represents a readout binary vector (128) generated by a bitwise OR operation between the first binary barcode (102A) and the second binary barcode (102B); comparing the readout binary vector (128) to a lookup table (200) generated based on a superimposed encoding, the lookup table configured to correlate the readout binary vector (128) to the first binary barcode (102A) and to the second binary barcode (102B); identifying a first human-readable identifier (204) (e.g., a name of farm or location in supply chain) associated with the first binary barcode (102A) a second human-readable identifier (204) associated with the second binary barcode (102B); and causing display of the first human-readable identifier and the second human-readable identifier.

Clause 2. The method of clause 1, wherein the first binary barcode and the second binary barcode are both binary vectors from (i.e., could be columns or rows) a Hadamard matrix (e.g., Sylvester-type).

Clause 3. The method of any one of clauses 1-2, wherein the first molecular taggant and the second molecular taggant comprise a plurality of sequence-defined polymers (e.g., DNA, proteins, plastics), each unique sequence-defined polymer associated with a single molecular bit.

Clause 4. The method of any one of clauses 1-3, wherein reading molecular taggants comprises contacting a substrate (e.g., paper ticket) with the molecular bits from the first molecular taggant and from the second molecular taggant collected from the (i) item or the (ii) mixed item, the substrate configured to have a plurality of spatially discrete regions (112) that each observably reacts to an individual one of the molecular bits (e.g., hybridization and fluorescence).

Clause 5. The method of any one of clauses 1-4, wherein the superimposed encoding creates a relationship between all valid binary barcodes such that the bitwise OR operation performed on any N (e.g., 2) valid binary barcodes generates a combined binary vector (208) that is different from all the valid binary barcodes and from all other combined binary vectors.

Clause 6. The method of any one of clauses 1-5, wherein the lookup table comprises a Hadamard matrix (covers but not limited to matrix with the T choose 2 OR operations appended).

Clause 7. The method of any one of clauses 1-6, wherein each entry (i.e., either a column or row) in the lookup table has at least a threshold Hamming distance from every other entry in the lookup table. (Could be broken into two claims)

Clause 8. The method of clause 7, wherein the threshold Hamming distance is at least twice an error rate of the error-prone readout.

Clause 9. A system for reading taggants on items comprising: a taggant reader (110) configured to generate an error-prone readout (114) (e.g., pattern of fluorescence on the ticket) of one or more molecular taggants (100) in a sample (108), the molecular taggants (100) using an encoding scheme comprising molecular bits (104) to indicate the value of a bit at a position (130) in a binary barcode (102); and a computing system (116) comprising a processing unit (802) and a memory (804), the memory storing: (could be local e.g., cell-phone and/or in the cloud) a readout analyzer (126) configured to convert the error-prone readout to a readout binary vector (128) having a length that is the same as the binary barcode (102); a lookup table (200) that is generated based on a superimposed encoding, the lookup table (200) comprising a plurality of binary vectors that each uniquely correlate to one or more binary barcodes; and instructions (816), executable on the processing unit (802), that cause the computing system (116) to compare the readout binary vector (128) to the lookup table (200) and identify a matching entry (210) in the lookup table (200) that is a one of the plurality of binary vectors that is the most similar (e.g., smallest Hamming distance) to the readout binary vector (128), the matching entry (210) corresponding to one or more binary barcodes.

Clause 10. The system of clause 9, wherein the taggant reader comprises a substrate (e.g., paper ticket) having a plurality of spatially discrete regions (112) that each observably reacts to an individual one of the molecular bits.

Clause 11. The system of any one of clauses 9-10, wherein the computing system comprises a handheld device (118) (e.g., smartphone) that also functions as a sensor (e.g., camera) to record reactions of the molecular bits with the taggant reader.

Clause 12. The system of any one of clauses 9-11, wherein the sample comprises molecular taggants from a first binary barcode and from a second binary barcode, the readout binary vector represents the result of a bitwise OR operation between the first binary barcode and the second binary barcode, and the matching entry corresponds to the first binary barcode and the second binary barcode.

Clause 13. The system of any one of clauses 9-12, wherein the lookup table includes all valid binary barcodes as binary vectors and the superimposed encoding creates a relationship between all the valid binary barcodes such that the bitwise OR operation performed on any N valid binary barcodes generates a binary vector that is different from all the valid binary barcodes and from the result of the bitwise OR operation on any other N valid binary barcodes.

Clause 14. The system of any one of clauses 9-13, wherein the lookup table comprises a Hadamard matrix (206) that has a dimension (e.g., height or width b/c square matrix) that is the same as a number of positions in the binary barcode. (e.g., if the barcode is 8 bits then the Hadamard matrix is 8×8.)

Clause 15. The system of clause 14, wherein the lookup table comprises the Hadamard matrix and concatenated thereto combined binary vectors (208) generated from bitwise OR operations performed on every pair of entries in the Hadamard matrix. (i.e., Hadamard with bitwise OR of T choose 2 appended)

Clause 16. The system of any one of clauses 9-15, wherein each binary vector in the lookup table has at least a threshold Hamming distance from all other binary vectors in the lookup table.

Clause 17. The system of any one of clauses 9-16, wherein the instructions further cause the computing system to cause display of one or more human-readable identifiers correlated with the one or more binary barcodes.

Clause 18. A method of creating molecular taggants for items comprising: creating a square, binary matrix (206) (e.g., Hadamard matrix) a with a property that every entry (i.e., column or row) in the matrix (206) is different and that a bitwise OR operation on any two entries in the matrix (206) creates a combined binary vector (208) that is different from each entry in the matrix (206) and from every other combined binary vector (208) created by the bitwise OR operation of any other two entries in the matrix (206), wherein at least a first entry in the matrix (206) corresponds to a binary barcode (102); (i.e., same order of 0s and 1s in a column as a barcode) associating a human-readable identifier (204) with the binary barcode (102) (e.g., Barcode Correspondence Table 202); assigning a different molecular bit (104) to each position in the binary barcode (102), each molecular bit (104) being a molecule that can be differentiated from all the other molecular bits (104) used in an encoding scheme i.e., deciding which DNA sequence represents each bit); synthesizing a plurality of molecules for the molecular bits (104) at each position in the binary barcode (102) (e.g., synthesis in bulk of every molbit); and creating a molecular taggant (100) (e.g., selecting which molbits to add to an item) that represents the binary barcode (102) according to the encoding scheme, the molecular taggant (100) including the molecular bit (104) corresponding to each position in the binary barcode (102) that has a first bit (i.e., 0 or 1) and omitting the molecular bit corresponding to each position in the binary barcode that has a second bit (i.e., the other one of 0 or 1).

Clause 19. The method of clause 18, wherein the matrix is a Hadamard matrix.

Clause 20. The method of at any one of clauses 18-19, wherein every entry in the matrix and every combined binary vector differs by at least a threshold Hamming distance.

Clause 21. The method of any one of clauses 18-20, further comprising: generating a plurality of molecular taggants, one for each entry in the matrix.

Clause 22. The method of any one of clauses 18-21, wherein the molecule is a sequence-defined polymer and each molecular bit is differentiated from all the other molecular bits based on the respective polymer sequences.

Clause 23. The method of any one of clauses 18-22, wherein the creating the molecular taggant comprises taking the molecular bits corresponding to each position in the binary barcode that has the first bit from pools of pre-synthesized molecular bits and combining the molecular bits.

Clause 24. The method of any one of clauses 18-23, further comprising: providing the molecular taggant to a user to apply to an item.

### CONCLUSION

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced. As used herein, “approximately” or “about” or similar referents denote a range of +10% of the stated value.

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

Furthermore, references have been made to publications, patents and/or patent applications throughout this specification. Each of the cited references is individually incorporated herein by reference for its particular cited teachings as well as for all that it discloses.

1. A method of tagging and reading taggants on items comprising:
applying a first molecular taggant that uses an encoding scheme comprising molecular bits to encode a first binary barcode to (i) an item or to (ii) a first item that will be included in a mixed item;
applying a second molecular taggant that uses the encoding scheme comprising the molecular bits to encode a second binary barcode to (i) the item or to (ii) a second item that will be included in the mixed item;
reading molecular taggants in a sample from (i) the item or the (ii) mixed item thereby generating an error-prone readout, wherein due to the presence encoding scheme the error-prone readout represents a readout binary vector generated by a bitwise OR operation between the first binary barcode and the second binary barcode;
comparing the readout binary vector to a lookup table generated based on a superimposed encoding, the lookup table configured to correlate the readout binary vector to the first binary barcode and to the second binary barcode;
identifying a first human-readable identifier associated with the first binary barcode a second human-readable identifier associated with the second binary barcode; and
causing display of the first human-readable identifier and the second human-readable identifier.

2. The method of claim 1, wherein the first binary barcode and the second binary barcode are both binary vectors from a Hadamard matrix.

3. The method of claim 1, wherein the first molecular taggant and the second molecular taggant comprise a plurality of sequence-defined polymers, each unique sequence-defined polymer associated with a single molecular bit.

4. The method of claim 1, wherein reading molecular taggants comprises contacting a substrate with the molecular bits from the first molecular taggant and from the second molecular taggant collected from the (i) item or the (ii) mixed item, the substrate configured to have a plurality of spatially discrete regions that each observably reacts to an individual one of the molecular bits.

5. The method of claim 1, wherein the superimposed encoding creates a relationship between all valid binary barcodes such that the bitwise OR operation performed on any N valid binary barcodes generates a combined binary vector that is different from all the valid binary barcodes and from all other combined binary vectors.

6. The method of claim 1, wherein the lookup table comprises a Hadamard matrix and each entry has at least a threshold Hamming distance from every other entry in the lookup table.

7. The method of claim 6, wherein the threshold Hamming distance is at least twice an error rate of the error-prone readout.

8. A system for reading taggants on items comprising:
a taggant reader configured to generate an error-prone readout of one or more molecular taggants in a sample, the molecular taggants using an encoding scheme comprising molecular bits to indicate the value of a bit at a position in a binary barcode; and
a computing system comprising a processing unit and a memory, the memory storing:
a readout analyzer configured to convert the error-prone readout to a readout binary vector having a length that is the same as the binary barcode;
a lookup table that is generated based on a superimposed encoding, the lookup table comprising a plurality of binary vectors that each uniquely correlate to one or more binary barcodes; and
instructions, executable on the processing unit, that cause the computing system to compare the readout binary vector to the lookup table and identify a matching entry in the lookup table that is a one of the plurality of binary vectors that is the most similar to the readout binary vector, the matching entry corresponding to one or more binary barcodes.

9. The system of claim 8, wherein the taggant reader comprises a substrate having a plurality of spatially discrete regions that each observably reacts to an individual one of the molecular bits.

10. The system of claim 8, wherein the sample comprises molecular taggants from a first binary barcode and from a second binary barcode, the readout binary vector represents the result of a bitwise OR operation between the first binary barcode and the second binary barcode, and the matching entry corresponds to the first binary barcode and the second binary barcode.

11. The system of claim 8, wherein the lookup table includes all valid binary barcodes as binary vectors and the superimposed encoding creates a relationship between all the valid binary barcodes such that the bitwise OR operation performed on any N valid binary barcodes generates a binary vector that is different from all the valid binary barcodes and from the result of the bitwise OR operation on any other N valid binary barcodes.

12. The system of claim 8, wherein the lookup table comprises a Hadamard matrix that has a dimension that is the same as a number of positions in the binary barcode.

13. The system of claim 12, wherein the lookup table comprises the Hadamard matrix and concatenated thereto combined binary vectors generated from bitwise OR operations performed on every pair of entries in the Hadamard matrix.

14. The system of claim 8, wherein each binary vector in the lookup table has at least a threshold Hamming distance from all other binary vectors in the lookup table.

15. A method of creating molecular taggants for items comprising:
creating a square, binary matrix a with a property that every entry in the matrix is different and that a bitwise OR operation on any two entries in the matrix creates a combined binary vector that is different from each entry in the matrix and from every other combined binary vector created by the bitwise OR operation of any other two entries in the matrix, wherein at least a first entry in the matrix corresponds to a binary barcode;
associating a human-readable identifier with the binary barcode;
assigning a different molecular bit to each position in the binary barcode, each molecular bit being a molecule that can be differentiated from all the other molecular bits used in an encoding scheme;
synthesizing a plurality of molecules for the molecular bits at each position in the binary barcode; and
creating a molecular taggant that represents the binary barcode according to the encoding scheme, the molecular taggant including the molecular bit corresponding to each position in the binary barcode that has a first bit and omitting the molecular bit corresponding to each position in the binary barcode that has a second bit.

16. The method of claim 15, wherein the matrix is a Hadamard matrix.

17. The method of claim 15, wherein every entry in the matrix and every combined binary vector differs by at least a threshold Hamming distance.

18. The method of claim 15, further comprising: generating a plurality of molecular taggants, one for each entry in the matrix.

19. The method of claim 15, wherein the molecule is a sequence-defined polymer and each molecular bit is differentiated from all the other molecular bits based on the respective polymer sequences.

20. The method of claim 15, wherein the creating the molecular taggant comprises taking the molecular bits corresponding to each position in the binary barcode that has the first bit from pools of pre-synthesized molecular bits and combining the molecular bits.
